(19) United States
(12) Patent Application Publication
(10) Pub. No.: US 2026/0307834 A1
(43) Pub. Date: Oct. 8, 2026

(54) Filling Process for Pharmaceutical Compositions

(71) Applicant: AMGEN INC., Thousand Oaks, CA (US)

(72) Inventors: Richard Crowther, Thousand Oaks, CA (US); Julian Jazayeri, Woodland Hills, CA (US); Seyed Reza Mirhassani Moghaddam, Thousand Oaks, CA (US); William Vega Vega, Thousand Oaks, CA (US)

(21) Appl. No.: 19/484,686

(22) PCT Filed: May 14, 2024
(86) PCT No.: PCT/US2024/029274

§ 371 Date: Nov. 14, 2025

Related U.S. Application Data
(60) Provisional application No. 63/466,440, filed on May 15, 2023.

(51) Int. Cl. B65B 3/00; B65B 3/04; B65B 3/28

(52) U.S. Cl. CPC B65B 3/003; B65B 3/04; B65B 3/28

(57) ABSTRACT

A method for reducing material usage in vessels is provided. The method includes obtaining historical fill weight data indicating actual, per-unit fill weight volumes for a plurality of vessels. The method also includes generating, based on analysis of the historical fill weight data, a distribution of simulated fill weights. The method further includes randomly sampling the distribution of simulated fill weights to select a subset of the simulated fill weights, and performing a quality check on the subset of the simulated fill weights. The method yet further includes generating a modified distribution of simulated fill weights at least by removing, from the distribution of simulated fill weights, samples of the subset that failed the quality check. The method also includes converting the modified distribution of simulated fill weights to a distribution of fill volumes, and calculating a distribution of deliverable volumes based on the distribution of fill volumes.

### CROSS-REFERENCE TO RELATED APPLICATION

Priority is claimed to U.S. Provisional Patent Application No. 63/466,440, filed May 15, 2023, the entire contents of which are hereby incorporated by reference herein.

### FIELD OF DISCLOSURE

The present application relates generally to filling vessels with a predetermined amount of material, and more specifically to reducing excess material usage when filling the vessels.

### BACKGROUND

Vessel filling processes are often tightly controlled to dispense a precise amount of a material (e.g., a fluid, a drug product, etc.) into a container (e.g., a syringe, a cartridge, an autoinjector, etc.). For example, “prefilled” syringes often contain an amount of a drug product that is determined based on specifications indicated on a drug product label (e.g., a deliverable volume of the drug product, a deliverable amount of the drug product, etc.). A fill target for an associated container filling process is often based on a type of material that is dispensed into the container, and the type of container.

As a particular example, a fill target for a syringe filling process is often based on a deliverable volume specified for a drug product that is dispensed into the syringe during a filling process. The fill target is typically greater than the specified deliverable volume to ensure that sufficient amount of a drug can be withdrawn for patient administration. To begin with, a “hold-up volume” is often added to the deliverable volume to account for drug product that may remain in the syringe subsequent to an associated injection. Moreover, an “excess amount” of a drug product is often added to the deliverable volume and the hold-up volume to account for other variables (e.g., fill nozzle data, fill process variations, pressure variations, temperature variations, etc.). Accordingly, a fill target is often equal to the deliverable volume, plus the hold-up volume, plus the excess volume.

As the material becomes less readily available (e.g., due to supply issues, high demand, high cost, etc.), unnecessary material usage becomes less tolerable. Correspondingly, as the material becomes less readily available, reducing a fill target for a vessel filling process becomes more desirable.

Accordingly, methods are needed for more efficient (e.g., less wasteful) material usage in vessel filling processes.

### SUMMARY

Embodiments described herein relate to methods for reducing excess material usage when filling vessels.

As described herein, a method for reducing material usage in vessels includes obtaining historical fill weight data indicating actual, per-unit fill weight volumes for a plurality of vessels. The method also includes generating, based on analysis of the historical fill weight data, a distribution of simulated fill weights. The method further includes randomly sampling the distribution of simulated fill weights to select a subset of the simulated fill weights, and performing a quality check on the subset of the simulated fill weights. The method yet further includes generating a modified distribution of simulated fill weights at least by removing, from the distribution of simulated fill weights, samples of the subset that failed the quality check. The method also includes converting the modified distribution of simulated fill weights to a distribution of fill volumes, and calculating a distribution of deliverable volumes based on the distribution of fill volumes. The method further includes causing a display to present one or both of (i) the distribution of deliverable volumes, and (ii) one or more quality performance metrics derived from the distribution of deliverable volumes.

A non-transitory computer-readable medium storing computer-readable instructions that, when executed by one or more processors, cause the one or more processors to obtain historical fill weight data indicating actual, per-unit fill weight volumes for a plurality of vessels. Further execution of the computer-readable instructions by the one or more processors, also causes the one or more processors to generate, based on analysis of the historical fill weight data, a distribution of simulated fill weights. Further execution of the computer-readable instructions by the one or more processors, also causes the one or more processors to randomly sample the distribution of simulated fill weights to select a subset of the simulated fill weights, and perform a quality check on the subset of the simulated fill weights. Further execution of the computer-readable instructions by the one or more processors, also causes the one or more processors to generate a modified distribution of simulated fill weights at least by removing, from the distribution of simulated fill weights, samples of the subset that failed the quality check. Further execution of the computer-readable instructions by the one or more processors, also causes the one or more processors to convert the modified distribution of simulated fill weights to a distribution of fill volumes, and calculate a distribution of deliverable volumes based on the distribution of fill volumes. Further execution of the computer-readable instructions by the one or more processors, also causes the one or more processors to cause a display to present one or both of (i) the distribution of deliverable volumes, and (ii) one or more quality performance metrics derived from the distribution of deliverable volumes.

Novel methods are provided for reducing material usage when filling vessels.

### BRIEF DESCRIPTION OF THE DRAWINGS

The skilled artisan will understand that the figures described herein are included for purposes of illustration and do not limit the present disclosure. The drawings are not necessarily to scale, and emphasis is instead placed upon illustrating the principles of the present disclosure. It is to be understood that, in some instances, various aspects of the described implementations may be shown exaggerated or enlarged to facilitate an understanding of the described implementations. In the drawings, like reference characters throughout the various drawings generally refer to functionally similar and/or structurally similar components.

FIG. 1 depicts a high level block diagram of an example system for reducing material usage in a vessel filling process.

FIG. 2 depicts an example method of generating a vessel filling process model.

FIG. 3 depicts a particular embodiment of the example method of FIG. 2.

FIG. 4 depicts an example method of using a vessel filling process model to generate a distribution of deliverable volumes and quality performance metrics derived from the distribution of deliverable volumes.

FIG. 5 depicts an example method of operating a vessel filling process based on a vessel filling process model.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercial feasible embodiment are often not depicted in order to facilitate a less obstructed view of these various embodiments. It will further be appreciated that certain actions and/or steps may be described or depicted in a particular order of occurrence while those skilled in the art will understand that such specificity with respect to sequence is not actually required. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### DETAILED DESCRIPTION

The various concepts introduced above and discussed in greater detail below may be implemented in any of numerous ways, and the described concepts are not limited to any particular manner of implementation. Examples of implementations are provided for illustrative purposes.

Methods and systems are provided for reducing material usage in a vessel filling process. The systems and methods generate a vessel filling process model (e.g., a mathematical model, a statistical model, a machine learning model, etc.) based on historical fill data associated with operation of the filling process. The systems and methods generate information (e.g., a distribution of deliverable volumes, a quality performance metric derived from a distribution of deliverable volumes, a fill target, etc.). A vessel filling process model may be used to determine whether a fill target will result in an acceptable risk (e.g., less than or equal to three parts per million, as determined using design for six-sigma, etc.) of an associated vessel filling process dispensing less than a threshold amount of material in each vessel.

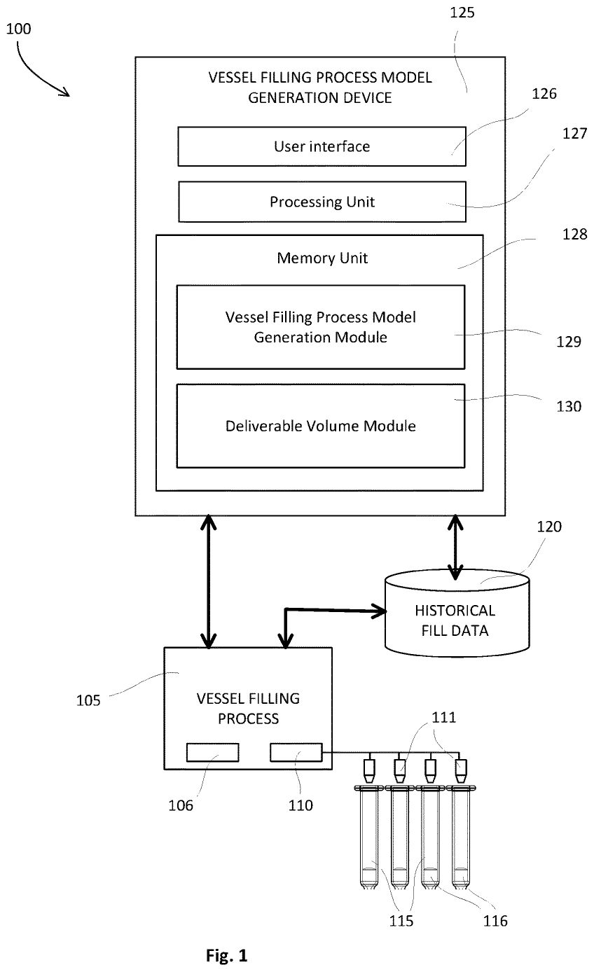

FIG. 1 depicts a high-level block diagram of an example system 100 for reducing material usage in a vessel filling process. The system 100 includes a vessel filling process model generation (VFPMG) device 125, a historical vessel fill database 120, and a vessel filling process 105. The vessel filling process 105 is implemented using a vessel fill module 106, a nozzle module 110 to fill a plurality of nozzles 111. Each nozzle 111 is configured to dispense a predetermined amount of material 116 (e.g., a drug product, etc.) into a respective vessel 115 (e.g., a hand-held autoinjector, a prefilled syringe, a prefilled cartridge, a prefilled autoinjector, a vial, etc.).

The vessel fill module 106 and the nozzle module 110 may be stored on the memory unit 128 as, for example, a set of computer-readable instructions that, when executed by the processing unit 127, cause the processing unit 127 to communicate vessel fill data and nozzle data between the vessel filling process 105, the VFPMG device 125, and the historical fill database 120. For example, the processing unit 127 may transmit fill target data to, and receive correlated vessel fill data from, the vessel filling process 105.

The historical vessel fill database 120 includes fill data (e.g., fill target data, vessel fill volume data, deliverable volume data, hold-up volume data, excess volume data, etc.) from in-process measurements for each filling nozzle 111. The historical vessel fill database 120 may also include nozzle data, vessel data, drug product data, material data, etc. For example, deliverable volume for a drug product is often specified in an associated drug product label. The deliverable volume of drug product is provided by a delivery mechanism such as, for example, a hand-held autoinjector, or a syringe. The deliverable volume typically corresponds to information specified on a drug product label, and the medication type, medication concentration, etc. A deliverable volume of a drug product may be, for example, 1.0 mL and a concentration of the drug product may be, for example, 100 mg/mL.

A hold-up volume is based on material characteristics of an associated material (e.g., a density of the material, a viscosity of the material, etc.) and delivery mechanism characteristics (e.g., a volume and/or discharge opening dimension of a syringe, autoinjector, etc.).

An excess fill weight is based on a specified deliverable volume (e.g., a deliverable volume specified on a drug product label, etc.), and a threshold increase in risk that an associated vessel filling process will not dispense the specified deliverable volume based on a particular fill target. A risk that a corresponding vessel filling process fails to satisfy associated standards (e.g., vessel filling process standards, six-sigma standards, etc.) is inversely proportional to an excess fill weight.

The VFPMG device 125 includes a user interface 126, a processing unit 127 (e.g., one or more processors, etc.), and a memory unit 128 (e.g., a non-transitory computer-readable medium, etc.). The memory unit 128 may include a vessel filling process model generation module 129 and a deliverable volume module 130 stored thereon as a set of computer-readable instructions that, when executed by the processing unit 127, causes the processing unit 127 to generate a vessel filling process model. For example, the processing unit 127 may generate hypothetical fill weight distributions for many lots (e.g., more than fifty, more than one thousand, more than one million, etc.) of a given drug product using a Monte Carlo simulation.

System 100 may implement algorithms that use fixed rules (e.g., acceptable risk that a vessel filling process will not dispense at least a threshold amount of material in each vessel, etc.). In addition to, or as an alternative to, a fixed rules-based model, the system 100 may implement various techniques relating to the training (and possibly validation and/or qualification) and/or use of one or more neural networks or other non-machine learning (ML) system to reduce material usage of a vessel filling process. The system 100 could also be used to test/qualify non-ML systems to reduce material usage.

For ease of explanation, system 100 is described herein as training and validating one or more vessel filling processes 105 using historical vessel fill data from historical fill data 120, and then using the trained/validated neural network(s) to determine, for example, a risk that a vessel filling process will not dispense a minimum amount of material in each vessel based upon a particular fill target. It is understood, however, that this need not be the case. For example, the training/validation may be performed by another system, and system 100 may then use the trained neural network(s) (e.g., during commercial production). In some embodiments, some or all of the historical fill data used for training and/or validation are generated using one or more offline (e.g., lab-based) “mimic stations” that closely replicate important aspects of commercial line equipment stations (e.g., vessels, material, vessel filling processes, nozzles, etc.), thereby expanding the training and/or validation library without causing excessive downtime of the commercial line equipment.

In some embodiments, however, system 100 includes two or more computers that are either co-located or remote from each other. In these distributed embodiments, the operations described herein relating to processing unit 127 and memory unit 128 may be divided among multiple processing units and/or memory units, respectively.

Processing unit 127 includes one or more processors, each of which may be a programmable microprocessor that executes software instructions stored in memory unit 128 to execute some or all of the functions of vessel filling process 105 as described herein. Processing unit 127 may include one or more central processing units (CPUs), for example. Alternatively, or in addition, some of the processors in processing unit 127 may be other types of processors (e.g., application-specific integrated circuits (ASICs), field-programmable gate arrays (FPGAs), etc.), and some of the functionality of system 100 as described herein may instead be implemented in hardware.

Memory unit 128 may include one or more volatile and/or non-volatile memories. Any suitable memory type or types may be included in memory unit 128, such as read-only memory (ROM), random access memory (RAM), flash memory, a solid-state drive (SSD), a hard disk drive (HDD), and so on. Collectively, memory unit 128 may store one or more software applications, the data received/used by those applications, and the data output/generated by those applications.

Memory unit 128 stores the software instructions of various modules that, when executed by processing unit 127, performs various functions for the purpose of training, validating, and/or qualifying one or more mathematical models, statistical model, artificial intelligence (Al) neural networks, etc. Specifically, in the example embodiment of FIG. 1, memory unit 128 stores the instructions of a vessel filling process model generation module 129 and a deliverable volume module 130. In other embodiments, memory unit 128 may not store one or both of modules 129, 130, and/or may store one or more other modules. In addition, or alternatively, one or both of modules 129, 130 may be implemented by a different computer system (e.g., a remote server coupled to system 100 via one or more wired and/or wireless communication networks). Moreover, the functionality of either or both of modules 129 and 130 may be divided among different software applications and/or computer systems. As just one example, in an embodiment where system 100 accesses a web service to train and use one or more vessel filling process models, the software instructions of vessel filling process model generation module 129 may be stored at a remote server.

Vessel filling process model generation module 129 comprises software that uses historical fill data 120 to train (generate and/or update) one or more models. Historical fill data 120 may be stored in memory unit 128, or in another local or remote memory (e.g., a memory coupled to a remote library server, etc.). In addition to training, module 129 may implement/run the model(s), e.g., by applying fill data acquired by vessel filling process 105 (or another vessel filling system) to the model(s), possibly after certain pre-processing is performed on the historical fill data as discussed below. In various embodiments, the model(s) trained and/or run by module 129 may determine a risk associated with a vessel filling process failing to dispense at least a threshold amount of material (e.g., a fill volume) in each vessel based on a particular fill target.

Module 129 may run the trained model(s) for purposes of validation, qualification, and/or inspection during commercial production. In one embodiment, for example, module 129 is used only to train and validate the model(s), and the trained model(s) is/are then transported to another computer system for qualification and inspection during commercial production (e.g., using another module similar to module 129). In some embodiments where vessel filling process model generation module 129 trains/runs multiple models, module 129 includes separate software for each model.

In some embodiments, deliverable volume module 130 controls/automates operation of vessel filling process 105 such that historical fill data 120 can be generated with little or no human interaction. Deliverable volume module 130 may cause a given vessel filling process 105 to capture vessel fill data by sending a command or other electronic signal (e.g., generating a pulse on a control line, etc.). Vessel filling process 105 may send the vessel fill data to VFPMG device 125, which may store the fill data in memory unit 128 for local processing. In alternative embodiments, vessel filling process 105 may be locally controlled, in which case deliverable volume module 130 may have less functionality than is described herein (e.g., only handling the retrieval of historical fill data), or may be omitted entirely from memory unit 128. The VFPMG device 125 may execute the vessel fill data module 106 and/or the nozzle data module 110 to implement local control.

A vessel filling process model (e.g., a mathematical model, a statistical model, etc.) may be combined with design for six sigma (DFSS) to optimize a deliverable volume at release testing for a commercial filling operation. The vessel filling process model uses historical fill weight data and release testing deliverable volume data to predict distributions of a fill volume and deliverable volume. The vessel filling process model determines a percent of vessels that fall under the deliverable volume specification limit. The model then simulates fill weight reductions to optimize the deliverable volume at release testing such that sufficient margin to the specification limit is maintained.

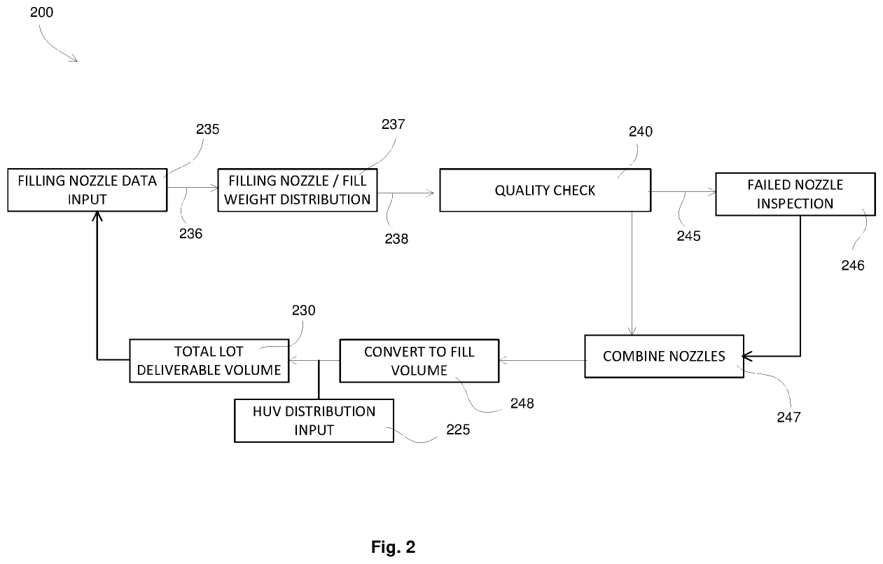

FIG. 2 depicts a method 200 of generating a vessel filling process model (e.g., a mathematical model, a statistical model, an Al model, etc.), which may be implemented by a processor (e.g., processing unit 127 of FIG. 1) executing, for example, at least a portion of the vessel filling process model generation module 129 and/or deliverable volume module 130. In particular, the vessel filling process model generation module 129 may receive a hold-up volume (HUV) distribution input (block 225). The HUV distribution input may be representative of, for example, fill weight data from in-process measurements for a plurality (millions) of previously filled vessels (samples), e.g., as represented by historical fill data 120.

The fill weight data from the in-process measurements may include a correlation with each respective nozzle 111. For example, historical fill weight data (indicating actual fill weights in past processes) may be collected on a per-fill nozzle basis (e.g., for 16 different nozzles, for one nozzle, etc.) over a number of lots or batches for a particular drug product.

The vessel filling process model generation module 129 may determine a total lot deliverable volume (block 230). For example, the vessel filling process model generation module 129 may multiply a per-vessel fill value (e.g., a fill volume, a fill weight, etc.) by a total number of vessels in a respective lot.

The vessel filling process model generation module 129 may receive filling nozzle data input (block 235). For example, the vessel filling process model generation module 129 may receive a distribution of means and standards of deviations for an amount of material discharged from each nozzle. The historical data 120 may include this received data, for example.

The vessel filling process model generation module 129 may generate a fill weight distribution (block 237). The historical data 120 may include this fill weight distribution data, for example. For example, the processing unit 127 may generate data 236 indicative of a fill weight distribution generated by running a Monte Carlo simulation. The fill weight distribution may include a correlation with material that is discharged from each nozzle 111. Based on this data, a statistical (Monte Carlo) model predicts a huge number of fill weight related results (e.g., millions) for each nozzle.

The vessel filling process model generation module 129 may segregate and inspect a subset 238 (e.g., two percent) of the samples from the distribution of fill weights2. The vessel filling process model generation module 129 may perform a fill weight data quality check on the subset 238 (block 240). For example, the vessel filling process model generation module 129 may virtually perform a quality control check that mirrors what would be physically done on a manufacturing line, such as randomly picking/selecting and then checking 2% of the randomly picked/selected samples/simulated values for each nozzle. The fill weight distribution may include a correlation of an amount of material discharged by each nozzle 111.

The vessel filling process model generation module 129 may generate a fail control signal 245, and any nozzle-associated data that fails the quality check may be inspected (block 246). For example, if a single sample is below a limit, a non-conforming (NC) counter may be triggered and the sample virtually removed.

The vessel filling process model generation module 129 may combine the nozzle-associated data that passed the quality check by aggregating the simulation data attributable to those nozzles (block 247). The vessel filling process model generation module 129 may convert a material weight to a fill volume for the aggregated simulation data attributable to the nozzle(s) that passed the quality check (block 248). For example, the vessel filling process model generation module 129 may divide a weight (mg) of a material by a density of a liquid that contains the material (mg/mL). In this example, a deliverable volume of a drug product is 1.0 mL when a fill weight of the drug product is 1.080 grams and the density of the liquid that contains the material is 1080 mg/mL.

The vessel filling process model generation module 129 may generate a distribution of deliverable volumes based on the vessel filling process model. Additionally, or alternatively, the vessel filling process model generation module 129 may generate a quality performance metric (e.g., a risk that an associated vessel filling process will not satisfy a specified deliverable volume, etc.) derived from a distribution of deliverable volumes based on the vessel filling process model.

A deliverable volume of a drug product may be, for example, 1.0 mL and a concentration of the drug product may be, for example, 100 mg/mL. A deliverable volume of a drug product may be, for example, 1.0 mL, and a fill weight of the drug product may be, for example, 1.080 grams. A vessel fill weight reduction of 0.015 grams may be verified to result in an acceptable risk using a vessel filling process model generated via method 200.

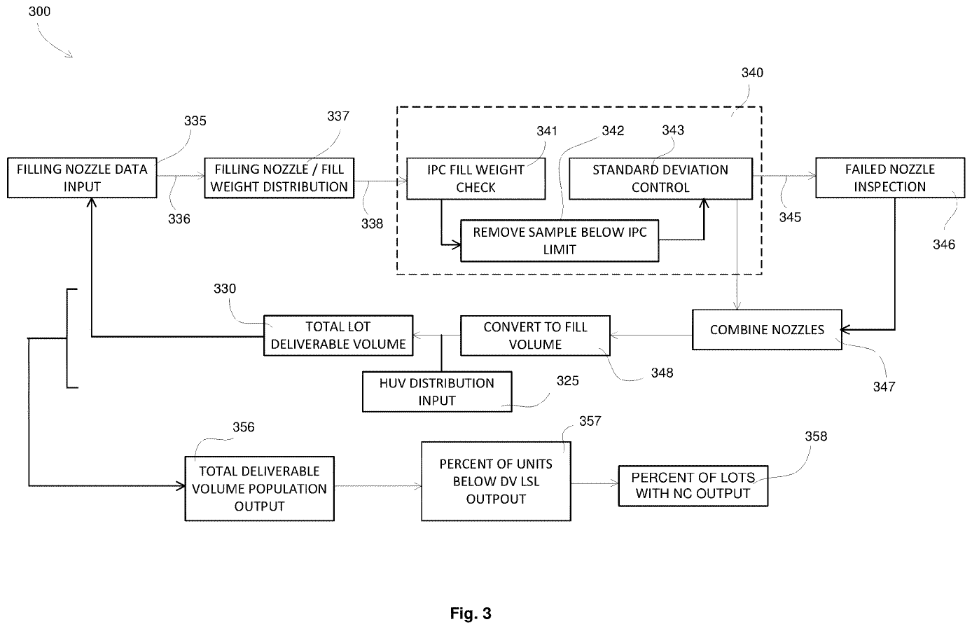

FIG. 3 depicts a particular embodiment of the method 200 of FIG. 2. In FIG. 3, a method 300 of generating vessel fill data uses a vessel filling process model (e.g., a mathematical model, a statistical model, an Al model etc.). The method 300 may be implemented by a processor (e.g., processing unit 127 of FIG. 1) executing, for example, at least a portion of the vessel filling process model generation module 129 and/or deliverable volume module 130. In particular, the vessel filling process model generation module 129 may receive a hold-up volume (HUV) distribution input (block 325). The HUV distribution input may be representative of, for example, fill weight data from in-process measurements for a plurality (millions) of previously filled vessels (samples), e.g., as represented by historical fill data 120. The HUV distribution input may be representative of, for example, fill weight data from in-process measurements. The fill weight data from the in-process measurements may include a correlation with each respective nozzle 111. Historical fill weight data (indicating actual fill weights in past processes) is collected on a per-fill nozzle basis (e.g., for 16 different nozzles, for one nozzle, etc.) over a number of lots or batches for a particular drug.

The vessel filling process model generation module 129 may determine a total lot deliverable volume (block 330). For example, the vessel filling process model generation module 129 may multiply a per-vessel fill value (e.g., a fill volume, a fill weight, etc.) by a total number of vessels in a respective lot.

The vessel filling process model generation module 129 may receive filling nozzle data input (block 335). For example, the vessel filling process model generation module 129 may receive a distribution of means and standards of deviations for an amount of material discharged from each nozzle. The historical data 120 may include data that is representative of the distribution of means and standards of deviations for an amount of material discharged from each nozzle.

The vessel filling process model generation module 129 may generate a fill weight distribution (block 337). The historical data 120 may include data that is representative of the fill weight distribution. For example, the processing unit 127 may generate a fill weight distribution based data 336 generated by running a Monte Carlo simulation. The fill weight distribution may include a correlation with material that is discharged from each nozzle 111. Based on this data, a statistical (Monte Carlo) model may predict a huge number of fill weight related results (e.g., millions) for each nozzle.

The vessel filling process model generation module 129 may segregate and inspect a subset 338 (e.g., two percent) of the samples from the distribution of fill weights 2. The vessel filling process model generation module 129 may perform a fill weight data quality check on the subset 338 (block 340). For example, the processing unit 127 may virtually perform a quality control check that mirrors an in-process control (IPC) fill weight check: first, 2% of the samples/predictions for each nozzle (e.g., sixteen nozzle, one nozzle, etc.) are randomly picked/sampled and checked (block 341). The fill weight distribution may include a correlation of an amount of material discharged by each nozzle 111. The processing unit 127 may virtually remove samples that are below an IPC threshold limit (block 342). The processing unit 127 may perform a standard deviation control (block 343).

The vessel filling process model generation module 129 may generate a fail control signal 345, and any nozzle-associated data that fails the quality check may be inspected (block 346). For example, if a single sample is below a limit, a non-conforming (NC) counter may be triggered and the sample virtually removed.

The vessel filling process model generation module 129 may combine the nozzle-associated data that passed the quality check by aggregating the simulation data attributable to those nozzles (block 347). The vessel filling process model generation module 129 may convert a material weight to a fill volume for the aggregated simulation data attributable to the nozzle(s) that passed the quality check (block 348). For example, the vessel filling process model generation module 129 may divide a weight (mg) of a material by a material density (mg/mL). For example, a deliverable volume of a drug product may be 1.0 mL and a fill weight of the drug product may be 1.114 grams.

The vessel filling process model generation module 129 may generate a total deliverable volume population output (block 356). The vessel filling process model generation module 129 may generate a percent of units below drug volume (DV) lower specification limit (LSL) output (block 357). The vessel filling process model generation module 129 may generate a percent of lots with non-conformance output (block 358).

The deliverable volume module 130 may generate a distribution of deliverable volumes based on the vessel filling process model. Additionally, or alternatively, the processing unit 127 may execute the deliverable volume module 130 to cause the processing unit 127 to, for example, generate a quality performance metric derived from a distribution of deliverable volumes based on the vessel filling process model.

A deliverable volume of a drug product may be, for example, 1.0 mL and a concentration of the drug product may be, for example, 100 mg/mL. A deliverable volume of a drug product may be, for example, 1.0 mL, and a fill weight of the drug product may be 1.080 grams. A vessel fill weight reduction of 0.015 grams, respectively, may be verified to result in an acceptable risk using a vessel filling process model generated using the method 300.

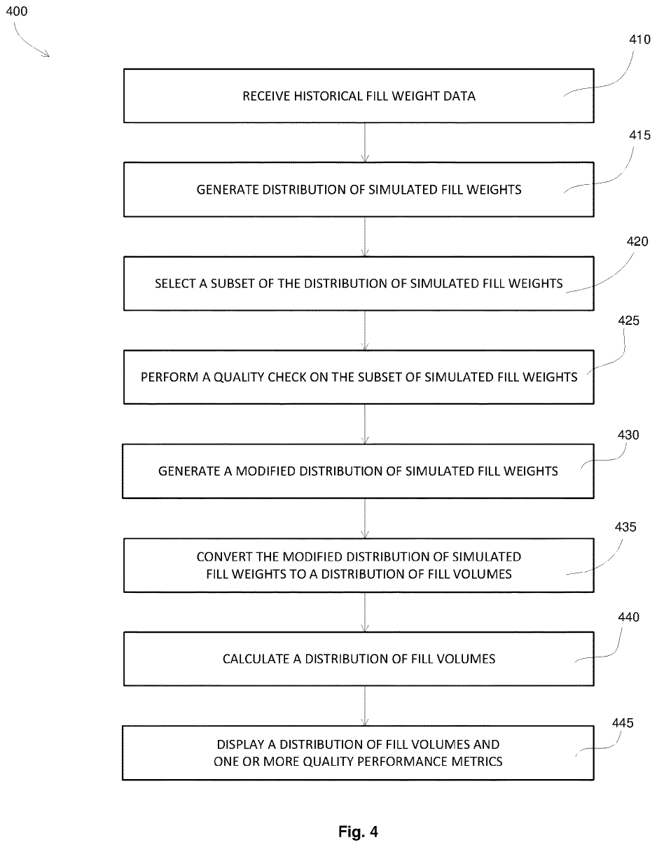

FIG. 4 is a method 400 of generating a distribution of deliverable volumes and a quality performance metric derived from a distribution of deliverable volumes based on a vessel filling process model (e.g., a statistical model, etc.). The method 400 may be implemented by a processor (e.g., processing unit 127 of FIG. 1) executing, for example, at least a portion of the vessel filling process model generation module 129 and/or deliverable volume module 130. In particular, the vessel filling process model generation module 129 may receive historical fill weight data (block 410). The HUV distribution input may be representative of, for example, fill weight data from in-process measurements. The fill weight data from the in-process measurements may include a correlation with each respective nozzle 111. Historical fill weight data (indicating actual fill weights in past processes) is collected on a per-fill nozzle basis (e.g., for 16 different nozzles, for one nozzle, etc.) over a number of lots or batches for a particular drug.

The vessel filling process model generation module 129 may generate a distribution of simulated fill weights (block 415). The vessel filling process model generation module 129 may select a subset of the distribution of simulated fill weights (block 420).

The vessel filling process model generation module 129 may perform a quality check on the subset of simulated fill weights (block 425). The vessel filling process model generation module 129 may generate a modified distribution of simulated fill weights (block 430). The vessel filling process model generation module 129 may convert the modified distribution of simulated fill weights to a distribution of fill volumes (block 435).

The vessel filling process model generation module 129 may calculate a distribution of fill volumes (block 440). The deliverable volume module 130 may display a distribution of fill volumes and one or more quality performance metrics (block 445).

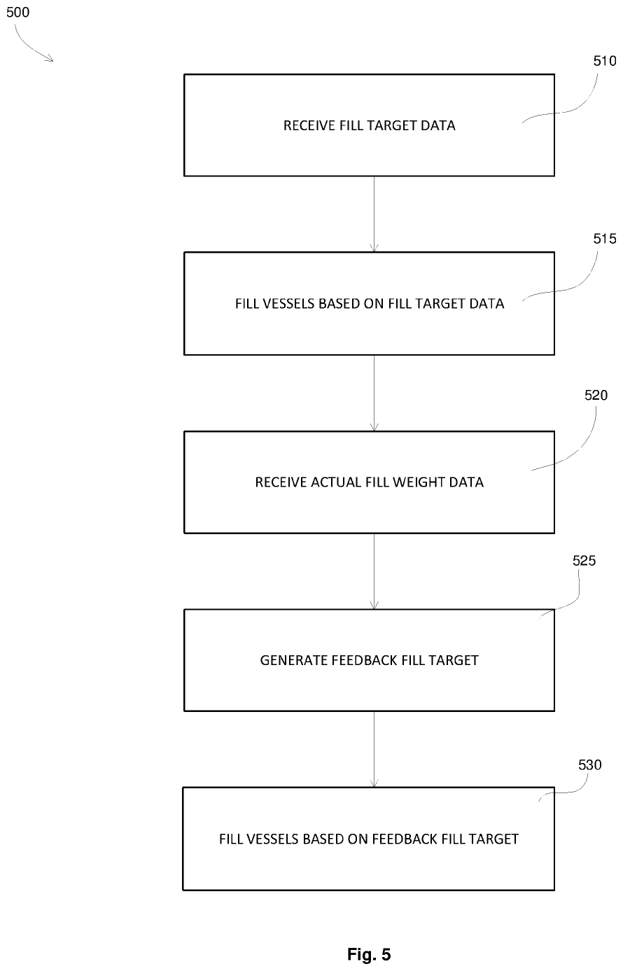

FIG. 5 depicts an example method 500 of operating a vessel fill process based on a vessel filling process model (e.g., a statistical model, etc.). The method 500 which may be implemented by a processor (e.g., processing unit 127 of FIG. 1) executing, for example, at least a portion of the vessel fill module 106 and/or the nozzle module 110. In particular, the vessel fill module 106 may receive fill target data (block 510).

The nozzle module 110 may fill vessels based on the fill target data (block 515). The vessel fill module 106 may receive actual fill weight data (block 520).

The vessel fill module 106 may generate a feedback fill target (block 525). The nozzle module 110 may fill vessels based on the feedback fill target (block 530).

Although the systems, methods, devices, and components thereof, have been described in terms of exemplary embodiments, they are not limited thereto. The detailed description is to be construed as exemplary only and does not describe every possible embodiment of the invention because describing every possible embodiment would be impractical, if not impossible. Numerous alternative embodiments could be implemented, using either current technology or technology developed after the filing date of this patent that would still fall within the scope of the claims defining the invention.

Those skilled in the art will recognize that a wide variety of modifications, alterations, and combinations can be made with respect to the above described embodiments without departing from the scope of the invention, and that such modifications, alterations, and combinations are to be viewed as being within the ambit of the inventive concept.

1. A method of filling a drug product in vessels, the method comprising:
obtaining historical fill weight data indicating actual, per-unit fill weight volumes for a plurality of vessels;
generating, based on analysis of the historical fill weight data, a distribution of simulated fill weights;
randomly sampling the distribution of simulated fill weights to select a subset of the simulated fill weights;
performing a quality check on the subset of the simulated fill weights;
generating a modified distribution of simulated fill weights at least by removing, from the distribution of simulated fill weights, samples of the subset that failed the quality check;
converting the modified distribution of simulated fill weights to a distribution of fill volumes;
calculating a distribution of deliverable volumes based on the distribution of fill volumes; and
causing a display to present one or both of (i) the distribution of deliverable volumes, and (ii) one or more quality performance metrics derived from the distribution of deliverable volumes.

2. The method of claim 1, wherein the analysis of the historical fill weight data includes a Monte Carlo analysis.

3. The method of claim 1, wherein the historical fill weight data includes a correlation of each vessel of the plurality of vessels with a respective nozzle associated with filling the vessel, and wherein the analysis of the historical fill weight data includes data indicating fill weight volumes on a per-nozzle basis.

4. The method of claim 3, wherein the quality check identifies at least one nozzle that is non-conforming.

5. The method of claim 4, wherein the quality check identifies as non-conforming all vessels associated with the at least one nozzle, and wherein the modified distribution of simulated fill weights excludes vessels associated with the at least one nozzle.

6. The method of claim 1, wherein the quality check includes determining whether a vessel fill level is below a minimum fill level threshold.

7. The method of claim 1, wherein the quality check includes determining whether a fill level standard of deviation, and determining whether the standard of deviation exceeds a fill level threshold.

8. The method of claim 1, wherein calculating the distribution of deliverable volumes includes generating the distribution of deliverable volumes based on (i) the distribution of fill volumes, and (ii) historical deliverable volume data or historical hold-up volume data.

9. The method of claim 1, further comprising:
adjusting, in real-time operation of a production line and based on the distribution of deliverable volumes, a target weight for a filling process of the production line.

10. A system for filling a drug product in vessels, the system comprising one or more processors and at least one memory having computer-readable instructions stored thereon that, when executed by the one or more processors, cause the one or more processors to perform the method of claim 1.

11. A non-transitory computer-readable medium having computer-readable instructions stored thereon that, when executed by one or more processors, cause the one or more processors to:
obtain historical fill weight data indicating actual, per-unit fill weight volumes for a plurality of vessels;
generate, based on analysis of the historical fill weight data, a distribution of simulated fill weights;
randomly sample the distribution of simulated fill weights to select a subset of the simulated fill weights;
perform a quality check on the subset of the simulated fill weights;
generate a modified distribution of simulated fill weights at least by removing, from the distribution of simulated fill weights, samples of the subset that failed the quality check;
convert the modified distribution of simulated fill weights to a distribution of fill volumes;
calculate a distribution of deliverable volumes based on the distribution of fill volumes; and
cause a display to present one or both of (i) the distribution of deliverable volumes, and (ii) one or more quality performance metrics derived from the distribution of deliverable volumes.

12. The non-transitory computer-readable medium as in claim 11, wherein the analysis of the historical fill weight data includes a Monte Carlo analysis.

13. The non-transitory computer-readable medium as in claim 11, wherein the historical fill weight data includes a correlation of each vessel of the plurality of vessels with a respective nozzle associated with filling the vessel, and wherein the analysis of the historical fill weight data includes data indicating fill weight volumes on a per-nozzle basis.

14. The non-transitory computer-readable medium as in claim 13, wherein the quality check identifies at least one nozzle that is non-conforming.

15. The non-transitory computer-readable medium as in claim 14, wherein the quality check identifies as non-conforming all vessels associated with the at least one nozzle, and wherein the modified distribution of simulated fill weights excludes vessels associated with the at least one nozzle.

16. The non-transitory computer-readable medium as in claim 11, wherein the quality check includes determining whether a fill level is below a minimum fill level threshold.

17. The non-transitory computer-readable medium as in claim 11, wherein the quality check includes determining whether a fill level standard of deviation, and determining whether the standard of deviation exceeds a fill level threshold.

18. The non-transitory computer-readable medium as in claim 11, wherein calculating the distribution of deliverable volumes includes generating the distribution of deliverable volumes based on (i) the distribution of fill volumes, and (ii) historical deliverable volume data or historical hold-up volume data.

19. The non-transitory computer-readable medium as in of claim 11, wherein further execution of the computer-readable instructions by the one or more processors causes the one or more processors to adjust, in real-time operation of a production line and based on the distribution of deliverable volumes, a target weight for a filling process of the production line.
